(19) United States
(12) Patent Application Publication
(10) Pub. No.: US 2021/0109102 A1
(43) Pub. Date: Apr. 15, 2021

(54) METHOD FOR PREDICTING THE RISK OF GETTING CANCER OR DIAGNOSING CANCER IN A FEMALE SUBJECT

(71) Applicant: SPHINGOTEC GMBH, Henningsdorf (DE)

(72) Inventors: Andreas BERGMANN, Berlin (DE); Olle MELANDER, Malmoe (SE)

(73) Assignee: SPHINGOTEC GMBH, Henningsdorf (DE)

(21) Appl. No.: 17/131,008

(22) Filed: Dec. 22, 2020

Related U.S. Application Data
(63) Continuation of application No. 14/383,428, filed on Sep. 5, 2014.
(60) Provisional application No. 61/608,350, filed on Mar. 8, 2012.

(30) Foreign Application Priority Data
Mar. 8, 2012 (EP) 12158679.6
Apr. 20, 2012 (EP) 12165054.3

(51) Int. Cl. G01N 33/574; G01N 33/74

(52) U.S. Cl. CPC G01N 33/57488; G01N 33/74; G01N 33/57415; G01N 2800/52; G01N 2333/726; G01N 2800/50; G01N 33/574

(57) ABSTRACT

Subject matter of the present invention is a method for predicting the risk of getting cancer in a female subject that does not suffer from cancer or alternatively diagnosing cancer in a female subject comprising determining the level of pro-neurotensin or fragments thereof of at least 5 amino acids in a bodily fluid obtained from said female subject; and correlating said level of pro-neurotensin or fragments thereof with the a risk for getting cancer, wherein an elevated level is predictive for an enhanced risk of getting cancer or alternatively diagnosing cancer wherein an elevated level is correlated with the diagnosis of cancer and, wherein said cancer is selected from the group comprising breast cancer, lung cancer, pancreatic cancer and colon cancer.

Subject matter of the present invention is a method for predicting the risk of getting cancer in a female subject that does not suffer from cancer or alternatively diagnosing cancer in a female subject comprising:
determining the level of pro-neurotensin or fragments thereof of at least 5 amino acids in a bodily fluid obtained from said female subject; and
correlating said level of pro-neurotensin or fragments thereof with a risk for getting cancer, wherein an elevated level is predictive for an enhanced risk of getting cancer or alternatively diagnosing cancer wherein an elevated level is correlated with the diagnosis of cancer and
   wherein said cancer is selected from the group comprising breast cancer, lung cancer, pancreatic cancer and colon cancer.

Neurotensin is a 13-amino acid neuropeptide derived from the prepro-neurotensin precursor and stochiometrically released together with the stable 117-amino acid peptide pro-neurotensin (P-NT) and the mature hormone binds to three different receptors, neurotensin receptor 1 and 2 (Ntsr1 and Ntsr2), which are G-protein coupled receptors and neurotensin receptor 3 (Ntsr3) which is non-G-protein coupled and also known as Sortillin-1 (SORT1).

Neurotensin is released peripherally from the small intestine as well as centrally from the hypothalamus. The peripheral secretion of neurotensin is stimulated by food-intake, especially by fat, and is known to regulate gastrointestinal motility and pancreatic and biliary secretion. Interestingly, neurotensin is implicated in appetite control as an anorectic hormone as it acutely reduces food intake following both central (intracerebroventricular) and peripheral (intraperitoneal) injection in rats, an effect which seems mainly mediated through the neurotensin-1 receptor (Ntsr1). In obese as compared to normal-weight human subjects, postprandial plasma neurotensin concentration was reduced following a liquid fatty meal (Widen et al 1992, Reg peptides; Plasma concentrations of regulatory peptides in obesity following modified sham feeding (MSF) and a liquid test meal), suggesting regulation of neurotensin secretion is disturbed in obesity. However, no large study has investigated if and how neurotensin is related to measures of obesity. Interestingly, P-NT significantly increases after gastric by-pass (Roux-en-Y), an operation shown to lead to normoglycemia in the majority of obese type 2 diabetes patients, but it is not known whether neurotensin is implicated in the development diabetes mellitus in general. Furthermore, the neurotensin system has been implicated in development of coronary artery disease and myocardial infarction as variation of the Ntsr3 (SORT1) gene is one of the strongest common coronary artery diseases susceptibility genes known in humans.

The mechanistic link between obesity and cancer is largely unknown, however, one of the dominating theories is that excess of fat deposits leads to increased peripheral aromatization of androgens and thus elevated circulating estrogen levels. In addition, one of the hallmarks of obesity, hyperinsulinemia, has been shown to inhibit hepatic production of sexual hormone binding globulin (SHBG), thus increasing bioavailable levels of both estrogens and androgens suggesting ways through which obesity may increase the risk of common forms of sex-hormone driven forms of cancer such as breast and prostate cancer. Interestingly, both neurotensin and Ntsr1 expression is common in malignant ductal breast cancer tumors and experimentally pharmacological blockade or RNA silencing of the NTSR1 reduces tumour growth in mice.

The level of expression of neurotensin receptor 1 (NTSR1) in breast cancer cells has been used for determining the prognosis of a subject suffering from breast cancer (US 2011/0305633). Further, it is stated in by the same authors that no clear correlation has been described today between circulating neurotensin and the stages of pancreas, prostate or medullar thyroid tumors probably due to rapid clearance by the liver. Interestingly, it was found that in a series of 51 patients with invasive ductal breast cancer 91% of all tumors were positive for neurotensin receptor 1 (NTSR1) but only 31% of all tumors were positive for neurotensin in said tissue (Souaze et al., Cancer Research (2006), 66:(12) pages 6243-6249).

There is some evidence that neurotensin and neurotensin receptors participate in cancer growth, in particular in lung cancer, pancreatic cancer and colon cancer (Carraway et al., Peptides (2006), 27 2445-2460). It has been reported that levels of NT in sera of patients with pancreatic cancer were significantly enhanced (Picheon et al., Anticancer Research (1999), 19 1445-50). Interestingly this group found that NT levels fell with progression of the disease for both prostate an pancreatic cancer. In contrast thereto, Meggiato et al. (Tumori (1996), 82; 592-5,) found that plasma levels of NT were normal in pancreatic cancer but elevated in case where pancreatitis was diagnosed.

The use of vasoactive peptides for prediction of cancer risks in males has been reported by Belting et al., Cancer, Epidemiology, Biomarkes & Prevention. MR-pro-ANP, MR-pro-ADM and copeptin was measured in the fasting plasma from participants of the Malmo Diet and Cancer Study that were free from cancer prior to the baseline exam in 1991 to 1994 (1768 males and 2293 females). The authors stated that among females, there was no relationship between biomarkers and cancer incidence.

A subject of the present invention was to investigate the prognostic and diagnostic power of NT for the prediction of cancer incidence and the prediction of the risk of reoccurrence of cancer. To address this issue, stable fragments of pro-neurotensin in fasting plasma were measured in said Swedish prospective cohort study (Malmo Diet and Cancer Study) and related baseline level of this biomarker to breast-cancer incidence during 15 years of follow-up.

Surprisingly, it has been shown that neurotensin is a powerful and highly significant biomarker for woman for predicting the risk of getting cancer in a female subject that does not suffer from cancer or alternatively diagnosing cancer in a female subject.

Thus, subject matter of the present invention is a method for predicting the risk of getting cancer in a female subject that does not suffer from cancer or alternatively diagnosing cancer in a female subject comprising:
a determining the level of pro-neurotensin or fragments thereof of at least 5 amino acids in a bodily fluid obtained from said female subject; and
correlating said level of pro-neurotensin or fragments thereof with the a risk for getting cancer, wherein an elevated level is predictive for an enhanced risk of getting cancer or alternatively diagnosing cancer wherein an elevated level is correlated with the diagnosis of cancer,
   wherein said cancer is selected from the group comprising breast cancer, lung cancer, pancreatic cancer and colon cancer.

Further, subject matter of the present invention is a method for predicting the risk of getting cancer in a female subject that does not suffer from cancer in a female subject comprising:
determining the level of pro-neurotensin 1-117 or fragments thereof of at least 5 amino acids or pro-neurotensin 1-117 comprising peptides in a bodily fluid obtained from said female subject; and
correlating said level of pro-neurotensin 1-117 or fragments thereof or pro-neurotensin 1-117 comprising peptides with the risk of getting cancer, wherein an elevated level is predictive for an enhanced risk of getting cancer and,
   wherein said cancer is selected from the group comprising breast cancer, lung cancer, pancreatic cancer and colon cancer.

In a specific embodiment of the invention said cancer is breast cancer.

The level of pro-neurotensin 1-117 or fragments thereof of at least 5 amino acids or pro-neurotensin 1-117 comprising peptides in a bodily fluid obtained from said female subject that is predictive for the risk of getting cancer in said female subject that does not suffer from cancer is released from the small intestine. It may not be released from cancer cells as the female subject does not suffer from cancer. The release of neurotensin from the small intestine is stimulated by food intake, especially by fat, and is known to regulate gastrointestinal motility and pancreatic and biliary secretion. Pro-neurotensin 1-117 and fragments thereof or pro-neurotensin 1-117 comprising peptides are used as a surrogate marker for the released neurotensin as neurotensin and pro-neurotensin 1-117 and fragments thereof or pro-neurotensin 1-117 comprising peptides are released in equimolar amounts from pro-neurotensin.

It is the surprising finding of the present invention that the peripheral secretion of neurotensin/pro-neurotensin 1-117 or fragments thereof of at least 5 amino acids or pro-neurotensin 1-117 comprising peptides is indicative for the susceptibility of a female subject to acquire cancer. Thus, dietary measures as reduction of that uptake may lower said risk in said female subject. It is known from literature that neurotensin and neurotensin receptor is overexpressed in tissue of cancer that is selected from the group comprising breast cancer, lung cancer, pancreatic cancer and colon cancer. Thus, elevated levels of neurotensin/pro-neurotensin 1-117 or fragments thereof of at least 5 amino acids or pro-neurotensin 1-117 comprising peptides in bodily fluids enhance the risk of getting a cancer where neurotensin and/or pro neurotensin plays a role, i. e. in cancers where neurotensin receptor is overexpressed in said cancer cells, e.g. in breast cancer, lung cancer, pancreatic cancer and colon cancer.

Thus, subject matter of the present invention is the determination of susceptibility of a woman to acquire cancer, e.g. breast cancer.

Data obtained in the present study revealed also a correlation between the risk of getting cancer in male subjects with the level of pro-neurotensin or fragments thereof of at least 5 amino acids in a bodily fluid obtained from said male subject; this correlation however, was not that significant for the present data set. Thus, there is a value for the method according to the invention also for male subjects but in the present study the observed effect was not as strong for males as compared to females.

The term “subject” as used herein refers to a living human or non-human organism. Preferably, herein the subject is a human subject.

The correlation between the level of pro-neurotensin 1-117 or fragments thereof of at least 5 amino acids or pro-neurotensin 1-117 comprising peptides in a bodily fluid obtained from said female subject and the risk of getting cancer is continuous, i.e. the higher the level the higher the risk. This can be seen from the data e.g. in Table 6. In comparison to the first quartile the second, third and fourth quartile exhibits higher Hazard Risks respectively.

For the sake of practicability the person skilled in the art may use threshold(s).

Thus, the term “elevated level” may mean a level above a threshold level.

In one embodiment of the invention the level of pro-neurotensin or fragments thereof of at least 5 amino acids or pro-neurotensin 1-117 comprising peptides in a bodily fluid is the fasting level of pro-neurotensin or fragments thereof of at least 5 amino acid or pro-neurotensin 1-117 comprising peptides. Fasting level means no food uptake 12 h prior blood sampling.

A bodily fluid may be selected from the group comprising blood, serum, plasma, urine, cerebro spinal liquid (csf), and saliva.

In one embodiment of the invention said female subject has never had a diagnosed cancer at the time the sample of bodily fluid is taken from said female subject.

In another embodiment said female subject has been diagnosed before with having cancer and has been cured at the time the sample of bodily fluid is taken from said female subject and the risk of reoccurrence of getting cancer is determined or alternatively the re-occurrence of cancer is diagnosed.

In one embodiment of the invention said female subject has been diagnosed as having at a cardiovascular disease or diabetes at the time the sample of bodily fluid is taken from said female subject.

Said cardiovascular disease may be selected from the group comprising heart failure, atherosclerosis and hypertension.

In another embodiment of the invention said female subject has been diagnosed as having at diabetes type 2 at the time the sample of bodily fluid is taken from said female subject.

The definition of diabetes is as follows: a history of physical diagnosis or being on anti-diabetic medication or having a fasting whole blood glucose>/=6.1 mmol/l (note this is =7.0 mmol/l in plasma) at the baseline examination.

The definition of normotensive/high blood pressure (HBP) is as follows:

HBP: Systolic BP>/=140 mmHg or Diastolic BP>/=90 mmHg or being on antihypertensive medications. Subjects having normal blood pressure (BP) are all other subjects, i.e subjects with Systolic BP<140 and Diastolic BP<90 and not being on antihypertensive medications.

The present data suggest a strong correlation between the level of pro-neurotensin or fragments thereof with cancer, in particular breast cancer, in woman with no prevalent diabetes, no prevalent breast cancer and no prevalent cardiovascular disease.

The present data also suggest a strong correlation between the level of pro-neurotensin or fragments thereof with cancer, in particular breast cancer, in hypertensive woman, which is a common high-risk group for cardiovascular disease.

Furthermore, the present data also suggest a strong correlation between the level of pro-neurotensin or fragments thereof with cancer, in particular breast cancer, in normotensive woman. Further, the present data suggest a strong correlation between the level of pro-neurotensin or fragments thereof with cancer, in particular breast cancer, in diabetic woman.

In a specific embodiment of the method according to the invention additionally at least one clinical parameter is determined selected from the group comprising: age, presence of diabetes mellitus, current smoking.

Fragments of pro-neurotensin that may be determined in a bodily fluid may be e.g. selected from the group of the following fragments:

(pro-neurotensin 1-147)
SEQ ID NO: 1
SDSEEEMKAL EADFLTNMHT SKISKAHVPS WKMTLLNVCS
LVNNLNSPAE ETGEVHEEEL VARRKLPTAL DGFSLEAMLT
IYQLHKICHS RAFQHWELIQ EDILDTGNDK NGKEEVIKRK
IPYILKRQLY ENKPRRPYIL KRDSYYY
(pro-neurotensin 1-125 (large neurotnedin N))
SEQ ID NO: 2
SDSEEEMKAL EADFLTNMHT SKISKAHVPS WKMTLLNVCS
LVNNLNSPAE ETGEVHEEEL VARRKLPTAL DGFSLEAMLT
IYQLHKICHS RAFQHWELIQ EDILDTGNDK NGKEEVI KR
KIPYIL
(neuromedin N:)
SEQ ID NO: 3
KIPYIL
(neurotensin)
SEQ ID NO: 4
pyroQLYENKPRRP YIL
(pro-neurotensin 1-117)
SEQ ID NO: 5
SDSEEEMKAL EADFLTNMHT SKISKAHVPS WKMTLLNVCS
LVNNLNSPAE ETGEVHEEEL VARRKLPTAL DGFSLEAMLT
IYQLHKICHS RAFQHWELIQ EDILDTGNDK NGKEEVI
(pro-neurotensin 1-132)
SEQ ID NO: 6
SDSEEEMKAL EADFLTNMHT SKISKAHVPS WKMTLLNVCS
LVNNLNSPAE ETGEVHEEEL VARRKLPTAL DGFSLEAMLT
IYQLHKICHS RAFQHWELIQ EDILDTGNDK NGKEEVIKRK
IPYILKRQLY EN
(pro-neurotensin 1-125)
Seq ID No 7:
SDSEEEMKAL EADFLTNMHT SKISKAHVPS WKMTLLNVCS
LVNNLNSPAE ETGEVHEEEL VARRKLPTAL DGFSLEAMLT
IYQLHKICHS RAFQHWELIQ EDILDTGNDK NGKEEVIKRK
IPYIL
(pro-neurotensin 120-140)
SEQ ID NO: 8
KIPYILKRQL YENKPRRPYI L
(pro-neurotcnsin 120-147)
SEQ ID NO: 9
KIPYILKRQL YENKPRRPYIL KRDSYYY
(pro-neurotensin 128-147)
SEQ ID NO: 10
QLYENKPRRP YILKRDSYYY

In amore specific embodiment of the method according to the present invention the level of pro-neurotensin 1-117 is determined.

In a specific embodiment the level of pro-neurotensin is measured with an immunoassay. More specifically an immunoassay is used as described in Ernst et al. (Peptides (2006), (27) 1787-1793). An immunoassay that may be useful for determining the level of pro-neurotensin or fragments thereof of at least 5 amino acids may comprise the steps as outlined in Example 2. All thresholds and values have to be seen in correlation to the test and the calibration used according to Example 2. A person skilled in the art may know that the absolute value of a threshold might be influenced by the calibration used. This means that all values and thresholds given herein are to be understood in context of the calibration used in herein (Example 2). A human P-NT-calibrator is available by ICI-Diagnostics, Berlin, Germany. Alternatively, the assay may also be calibrated by synthetic or recombinant P-NT 1-117 or fragments thereof (see also Ernst et. al, 2006).

The threshold for determining the risk of getting breast cancer in a female subject or diagnosing breast cancer in a female subject according to the methods of the present invention is above 78 pmol/l PNT, preferred 100 pmol/l, more preferred 150 pmol/1. In a specific embodiment said threshold is about 100 pmol/l. These thresholds are related to the above mentioned calibration method. A P-NT value above said threshold means that the subject has an enhanced risk of getting cancer or has already cancer.

In one embodiment of the invention said method is performed more than once in order to monitor the risk of getting breast cancer in a female subject or in order to monitor the course of treatment. In one specific embodiment said monitoring is performed in order to evaluate the response of said female subject to preventive and/or therapeutic measures taken.

In one embodiment of the invention the method is used in order to stratify said female subjects into risk groups.

Subject of the present invention is also a method for predicting the risk of getting cancer in a female or identifying a female subject having an enhanced risk for getting cancer according to any of the preceding embodiments, wherein the level of pro-neurotensin or fragments thereof of at least 5 amino acids in a bodily fluid obtained from said female subject either alone or in conjunction with other prognostically useful laboratory or clinical parameters is used for the prediction of a subject's risk for getting an adverse event by a method which may be selected from the following alternatives:
Comparison with the median of the level of pro-neurotensin or fragments thereof of at least 5 amino acids in a bodily fluid obtained from said female subject in an ensemble of pre-determined samples in a population of “healthy” or “apparently healthy” subjects,
Comparison with a quantile of the level of pro-neurotensin or fragments thereof of at least 5 amino acids in a bodily fluid obtained from said female subject in an ensemble of pre-determined samples in a population of “healthy” or “apparently healthy” subjects,
Calculation based on Cox Proportional Hazards analysis or by using Risk index calculations such as the NRI (Net Reclassification Index) or the IDI (Integrated Discrimination Index).

According to the method of the present invention said female subject's risk for developing cancer may be reclassified as a consequence of the determination of the level of pro-neurotensin 1-117 or fragments thereof of at least 5 amino acids or pro-neurotensin 1-117 comprising peptides in a bodily fluid obtained from said female subject.

Subject matter of the invention is also point-of-care device for performing a method according to the invention.

Subject matter of the invention is also a binder to neurotensin or to a neurotensin receptor, for the use in prevention or therapy of cancer in a female subject.

In one embodiment of the invention the binder reduces the bioactivity of neurotensin to 70% or less.

In one embodiment of the invention the cancer is selected from the group comprising breast cancer, lung cancer, pancreatic cancer and colon cancer.

According to the invention the binder to neurotensin is selected from the group consisting of antibodies e.g. IgG, a typical full-length immunoglobulin, or antibody fragments containing at least the F-variable domain of heavy and/or light chain as e.g. chemically coupled antibodies (fragment antigen binding) including but not limited to Fab-fragments including Fab minibodies, single chain Fab antibody, monovalent Fab antibody with epitope tags, e.g. Fab-V5Sx2; bivalent Fab (mini-antibody) dimerized with the CH3 domain; bivalent Fab or multivalent Fab, e.g. formed via multimerization with the aid of a heterologous domain, e.g. via dimerization of dHLX domains, e.g. Fab-dHLX-FSx2; F(ab′)2-fragments, scFv-fragments, multimerized multivalent or/and multispecific scFv-fragments, bivalent and/or bispecific diabodies, BITE® (bispecific T-cell engager), trifunctional antibodies, polyvalent antibodies, e.g. from a different class than G; single-domain antibodies, e.g. nanobodies derived from camelid or fish immunoglobulines.

According to the invention the binder to a neurotensin receptor is selected from the group consisting of antibodies e.g. IgG, a typical full-length immunoglobulin, or antibody fragments containing at least the F-variable domain of heavy and/or light chain as e.g. chemically coupled antibodies (fragment antigen binding) including but not limited to Fab-fragments including Fab minibodies, single chain Fab antibody, monovalent Fab antibody with epitope tags, e.g. Fab-V5Sx2; bivalent Fab (mini-antibody) dimerized with the CH3 domain; bivalent Fab or multivalent Fab, e.g. formed via multimerization with the aid of a heterologous domain, e.g. via dimerization of dHLX domains, e.g. Fab-dHLX-FSx2; F(ab′)2-fragments, scFv-fragments, multimerized multivalent or/and multispecific scFv-fragments, bivalent and/or bispecific diabodies, BITE® (bispecific T-cell engager), trifunctional antibodies, polyvalent antibodies, e.g. from a different class than G; single-domain antibodies, e.g. nanobodies derived from camelid or fish immunoglobulines, or a peptide antagonist e.g. [D-Trp¹¹]-Neurotensin, [Tyr(Me)¹¹]-Neurotensin (e.g. described by Quiron et al.), or a non-peptide antagonist, e.g. Levocabastine, SR-48692 (NTS1 selective), SR-142948 (unselective), SR-142948A, CP 96345, [3H]SR-48692, SR 48692, SR-48527 and SR-49711, or a binder scaffold e.g. tetranectin-based non-Ig scaffolds (e.g. described in US 2010/0028995), fibronectin scaffolds (e.g. described in EP 1266 025; lipocalin-based scaffolds ((e.g. described in WO 2011/154420); ubiquitin scaffolds (e.g. described in WO 2011/073214), transferring scaffolds (e.g. described in US 2004/0023334), protein A scaffolds (e.g. described in EP 2231860), ankyrin repeat based scaffolds (e.g. described in WO 2010/060748), microproteins preferably microproteins forming a cystine knot) scaffolds (e.g. described in EP 2314308), Fyn SH3 domain based scaffolds (e.g. described in WO 2011/023685) EGFR-A-domain based scaffolds (e.g. described in WO 2005/040229) and Kunitz domain based scaffolds (e.g. described in EP 1941867).

Another preventive measure may be the reduction of neurotensin secretion e.g. by watching a low fat diet or other dietary measurements that may reduce neurotensin secretion.

### EXAMPLES

### Example 1

### Development of Antibodies

### Peptides/Conjugates for Immunization:

Peptides for immunization were synthesized (JPT Technologies, Berlin, Germany) with an additional N-terminal Cystein residue for conjugation of the peptides to bovine serum albumin (BSA). The peptides were covalently linked to BSA by using SulfoLink-Coupling gel (Perbio-science, Bonn, Germany). The coupling procedure was performed according to the manual of Perbio.

Labelled antibody (LA) peptide (P-NT 1-19):
H-CSDSEEEMKALEADFLTNMH-NH2
Solid phase antibody (SPA) peptide (P-NT 44-62):
H-CNLNSPAEETGEVHEEELVA-NH2

The antibodies were generated according to the following method:

A BALB/c mouse were immunized with 100 μg peptide-BSA-conjugate at day 0 and 14 (emulsified in 100 μl complete Freund's adjuvant) and 50 μg at day 21 and 28 (in 100 μl incomplete Freund's adjuvant). Three days before the fusion experiment was performed, the animal received 50 μg of the conjugate dissolved in 100 μl saline, given as one intraperitonal and one intra venous injection.

Splenocytes from the immunized mouse and cells of the myeloma cell line SP2/0 were fused with 1 ml 50% polyethylene glycol for 30 s at 37° C. After washing, the cells were seeded in 96-well cell culture plates. Hybrid clones were selected by growing in HAT medium [RPMI 1640 culture medium supplemented with 20% fetal calf serum and HAT-supplement]. After two weeks the HAT medium is replaced with HT Medium for three passages followed by returning to the normal cell culture medium.

The cell culture supernatants were primary screened for antigen specific IgG antibodies three weeks after fusion. The positive tested microcultures were transferred into 24-well plates for propagation. After retesting the selected cultures were cloned and recloned using the limiting-dilution technique and the isotypes were determined.

(Lane, R. D. “A short-duration polyethylene glycol fusion technique for increasing production of monoclonal antibody-secreting hybridomas”, J. Immunol. Meth. 81: 223-228; (1985), Ziegler, B. et al. “Glutamate decarboxylase (GAD) is not detectable on the surface of rat islet cells examined by cytofluorometry and complement-dependent antibody-mediated cytotoxicity of monoclonal GAD antibodies”, Horm. Metab. Res. 28: 11-15, (1996)).

### Monoclonal Antibody Production

Antibodies were produced via standard antibody production methods (Marx et al., Monoclonal Antibody Production (1997), ATLA 25, 121) and purified via Protein A-chromatography. The antibody purities were >95% based on SDS gel electrophoresis analysis.

### Example 2

### Immunoassay for the Quantification of Human Pro-Neurotensin

The technology used was a sandwich coated tube luminescence immunoassay, based on Acridinium ester labelling.

Labelled compound (tracer): 100 μg (100 μl) LA (1 mg/ml in PBS, pH 7.4, was mixed with 10 μl Acridinium NHS-ester (1 mg/ml in acetonitrile, InVent GmbH, Germany) (EP 0353971) and incubated for 20 min at room temperature. Labelled LA was purified by gel-filtration HPLC on Bio-Sil SEC 400-5 (Bio-Rad Laboratories, Inc., USA) The purified LA was diluted in (300 mmol/1 potassiumphosphate, 100 mmol/NaCl, 10 mmol/1 Na-EDTA, 5 g/l bovine serum albumin, pH 7.0). The final concentration was approx. 800.000 relative light units (RLU) of labelled compound (approx. 20 ng labeled antibody) per 200 μl. Acridiniumester chemiluminescence was measured by using an AutoLumat LB 953 (Berthold Technologies GmbH & Co. KG).

Solid phase: Polystyrene tubes (Greiner Bio-One International AG, Austria) were coated (18 h at room temperature) with SPA (1.5 μg SPA/0.3 ml 100 mmol/l NaCl, 50 mmol/l Tris/HCl, pH 7.8). After blocking with 5% bovine serum albumine, the tubes were washed with PBS, pH 7.4 and vacuum dried.

### Calibration:

The assay was calibrated, using dilutions of P-NT-containing human serum. A pool of human sera with high P-NT-immunoreactivity (InVent Diagostika, Hennigsdorf, Germany) was diluted with horse serum (Biochrom AG, Deutschland) (assay standards).

The standards were calibrated by use of the human ProNT-calibrator (ICI-Diagnostics, Berlin, Germany). Alternatively, the assay may be calibrated by synthetic or recombinant P-NT 1-117 or fragments thereof (see also Ernst et al., 2006).

### ProNT Immunoassay:

50 μl of sample (or calibrator) was pipetted into SPA coated tubes, after adding labelled LA (200 ul), the tubes were incubated for 16-22 h at 18-25° C. Unbound tracer was removed by washing 5 times (each I ml) with washing solution (20 mmol/l PBS, pH 7.4, 0.1% Triton X-100).

Tube-bound LA was measured by using the LB 953.

FIG. 1 shows a typical P-NT dose/signal curve.

### Example 3

### Population Study

### Methods

We measured P-NT in fasting plasma from 2559 female participants of the population based Malmö Diet and Cancer Study baseline exam in 1991-1994 (men age 58±6 years and 59% females). We used multivariable adjusted (all traditional cardiovascular risk factors, diabetes risk factors and in analyses of cancer also heredity for cancer) Cox proportional hazards models to relate baseline P-NT (hazard ratio per each standard deviation increase of log-transformed P-NT) to the time to the first event of each of the studied endpoints during a median follow-up time of more than 12 years. Endpoints were retrieved through the Swedish National Hospital Discharge Registry, the Swedish Myocardial Infarction Registry, the Stroke in Malmö Registry and the Swedish Cancer Registry. Retrieval of endpoints through these registries has been validated and found to be accurate (see also Belting et al Cancer Epidemiol Biomarkers Prev; 1-10._2012 AACR).

Clinical Characteristics of Females in the Study

TABLE 1
Descriptive Statistics

| | N | Mean | Std. Deviation |
|---|---|---|---|
| Age at MDCS screening | 2559 | 57.554 | 5.9403 |
| Systolic blood pressure (mmHg) | 2559 | 140.50 | 19.311 |
| Diastolic blood pressure (mmHg) | 2559 | 85.65 | 9.117 |
| body-mass-index (weight/kgxkg) | 2559 | 25.5196 | 4.19083 |
| WAIST (cm) | 2559 | 76.99 | 10.245 |
| Glucose (mmol/l) | 2559 | 5.0418 | 1.21798 |
| Triglycerides (mmol/l) | 2559 | 1.2245 | .58404 |
| High density lipoprotein (mmol/l) | 2559 | 1.5123 | .36949 |
| Low density lipoprotein (mmol/l) | 2559 | 4.2016 | 1.04762 |
| P-INSULIN | 2512 | 7.223 | 5.4223 |
| P-NT (pmol/l) | 2559 | 125.60633 | 77.681673 |

TABLE 2
Q + Diary: Anti hypertension treatment
(C02, C03, C07, C08, C09)
at baseline according to questionnaire or diary book

| | | | Valid | Cumulative |
|---|---|---|---|---|
| | Frequency | Percent | Percent | Percent |
| Valid No | 2173 | 84.9 | 84.9 | 84.9 |
| Yes | 386 | 15.1 | 15.1 | 100.0 |
| Total | 2559 | 100.0 | 100.0 | |

TABLE 3
DIAB MELL (fb >6.0 mmol/l or pos Q DM)

| | Frequency | Percent | Valid Percent | Cumulative Percent |
|---|---|---|---|---|
| Valid no | 2396 | 93.6 | 93.6 | 93.6 |
| yes | 163 | 6.4 | 6.4 | 100.0 |
| Total | 2559 | 100.0 | 100.0 | |

TABLE 4
current_smoker0

| | | | | Cumulative |
|---|---|---|---|---|
| | Frequency | Percent | Valid Percent | Percent |
| Valid .00 | 1906 | 74.5 | 74.5 | 74.5 |
| 1.00 | 653 | 25.5 | 25.5 | 100.0 |
| Total | 2559 | 100.0 | 100.0 | |

TABLE 5
QUARTILES OF PNT IN WOMEN:
PNT (pmol/l)

| Percentile group | | | | |
|---|---|---|---|---|
| of PNT (pmol/l) | N | Median | Minimum | Maximum |
| 1 | 639 | 62.37000 | 5.100 | 78.580 |
| 2 | 639 | 92.07000 | 78.610 | 108.770 |
| 3 | 641 | 125.07000 | 108.960 | 150.000 |
| 4 | 640 | 194.38500 | 150.050 | 1154.520 |
| Total | 2559 | 108.96000 | 5.100 | 1154.520 |

Quartile-concentrations were almost identical in all shown subgroup analysis.

### P-NT and Prediction of Breast Cancer

We assessed the relationship between P-NT and breast cancer (Table 6). There was a strong relationship between P-NT and breast cancer in females. In a fully adjusted model each SD increase of P-NT was associated with a 35% increased risk of future breast cancer and the top versus bottom quartile of P-NT identified a more than 2-fold difference in risk of breast cancer (see table 6 and table 7).

TABLE 6
BREAST CANCER

| | HR per | | Quartile | Quartile | Quartile | Quartile | P for |
|---|---|---|---|---|---|---|---|
| | 1 SD | P-value | 1 | 2 | 3 | 4 | trend |
| Women | 1.36 | <0.001 | 1.0 | 1.20 | 1.55 | 2.08 | 0.002 |
| (2140/ | (1.14- | | (ref) | (0.69- | (0.93- | (1.27- | |
| 135) | 1.59) | | | 2.07) | 2.61) | 3.42) | |

Multivariate Cox proportional Hazards models for baseline P-NT versus incidence of breast cancer

TABLE 7
BREAST CANCER-FEMALES
(females with any cancer history were excluded, 2140 women/135 events)

| | | | | | | | 95.0% CI for | |
|---|---|---|---|---|---|---|---|---|
| | | | | | | | Exp(B) | |
| Variables in the equation | B | SE | Wald | df | Sig. | Exp(B) | Lower | Upper |
| AGE | .004 | .017 | .058 | 1 | .810 | 1.004 | .971 | 1.039 |
| AHT_B | −.014 | .262 | .003 | 1 | .958 | .986 | .590 | 1.648 |
| SBP_B | −.001 | .005 | .013 | 1 | .911 | .999 | .989 | 1.010 |
| BMI_B | .046 | .025 | 3.230 | 1 | .072 | 1.047 | .996 | 1.100 |
| HDL_B | −.065 | .263 | .061 | 1 | .805 | .937 | .560 | 1.568 |
| LDL_B | −.080 | .093 | .752 | 1 | .386 | .923 | .769 | 1.107 |
| current_smoker0 | .298 | .201 | 2.202 | 1 | .138 | 1.347 | .909 | 1.996 |
| LNINS | −.368 | .204 | 3.234 | 1 | .072 | .692 | .464 | 1.034 |
| DM_B | −.103 | .413 | .062 | 1 | .803 | .902 | .401 | 2.028 |
| HER_CANCER_0 | .014 | .179 | .006 | 1 | .936 | 1.015 | .714 | 1.441 |
| ZLN_PNT | .307 | .087 | 12.520 | 1 | .000 | 1.360 | 1.147 | 1.612 |

FIG. 2: Kaplan Meier graphs, illustrating the cumulative breast cancer diagnosis in women a) below and above P-NT median (109 pmol/l) and b) quartile (Q) 1 vs quartile 4 (below 79 pmol/l and above 150 pmol/l)

Legend FIGS. 2a and 2b: Increased P-NT indicates a long term increased risk of breast cancer development. Since any women with cancer history at day of baseline (blood sampling) were excluded, PNT is highly predictive for future breast cancer development. Over all, women from Q 4 have an 2 times higher risk to develop breast cancer than women from Q 1.

Subgroup Analysis in Women with and without Diabetes/High Blood Pressure at Baseline.

Definition Diabetes: A history of physical diagnosis or being on anti-diabetic medication or having a fasting whole blood glucose>/=6.1 mmol/1 (note this is =7.0 mmol/1 in plasma) at the baseline examination.

Definition normotensive/high blood pressure (HBP):

HBP: Systolic BP>/=140 mmHg or Diastolic BP>/=90 mmHg or being on antihypertensive medications. Normal BP: all other subjects.

Using the same variables in the equation, we investigated different subgroups of women for prediction of cancer (women with any cancer history were excluded): Table 8

TABLE 8

| | No of | No of | Hazard risk | Significance |
|---|---|---|---|---|
| Subgroup | subjects | events | per 1SD PNT | (p-value) |
| all | 2140 | 135 | 36% | <0.001 |
| HBP Women | 1294 | 79 | 26.3% | <0.05 |
| Normotensive | 846 | 56 | 52% | 0.001 |
| women | | | | |
| Women w/o | 1984 | 128 | 35% | <0.001 |
| history of | | | | |
| CV-events | | | | |
| and w/o | | | | |
| Diabetes | | | | |
| Normotensive | 891 | 52 | 45% | 0.006 |
| women w/o | | | | |
| history of | | | | |
| CV-events | | | | |
| and w/o | | | | |
| Diabetes | | | | |

In all subgroups P-NT is significantly predictive for breast cancer development. The predictive power of PNT was slightly stronger in healthier women.

### Example 4

Reclassification of Woman into Risk Groups

### Methods:

We calculated model c-statistics and reclassification across 10-year predicted risk categories for the different events (<5%, >=5-10%, >=10-20% and >=20%, respectively) with Net Reclassification Improvement (NRI) for models with and without P-NT.
Pencina M J, D'Agostino R B. Overall C as a measure of discrimination in survival analysis: model specific population value and confidence interval estimation. Stat Med. Jul. 15, 2004; 23(13):2109-2123.
Pencina M J, D'Agostino R B, Sr., D'Agostino R B, Jr., Vasan R S. Evaluating the added predictive ability of a new marker: from area under the ROC curve to reclassification and beyond. Stat Med. Jan. 30, 2008; 27(2):157-172; discussion 207-112.
Ridker P M, Buring J E, Rifai N, Cook N R. Development and validation of improved algorithms for the assessment of global cardiovascular risk in women: the Reynolds Risk Score. Jama. Feb. 14, 2007; 297(6):611-619.

All analyses were performed with Stata software version 11 (StataCorp, College Station, Tex.).

A two-sided P-value of <0.05 was considered statistically significant.

### Results:

### Discrimination and Risk Reclassification in Females

Adding P-NT on top of all breast cancer risk factors, significantly correctly reclassified women across categories of risk of 10 year breast cancer outcome with an NRI of 25%. Of women who later developed breast cancer, 34% were moved to a higher category of risk whereas 27% of women who did not develop breast cancer were moved to a lower category of risk. The C-statistic for breast cancer increased after adding P-NT on top of known breast cancer risk factors by 4.7% (Table 9).

| Breast cancer | | |
|---|---|---|
| (n = 1657, 113 | | |
| events) | C-statistic | NRI |
| Basic model¶ | 61.4% | ref |
| +pro-neurotensin | 66.1% | 25% (P < 0.001) |

*Clinical NRI refers to correct movements up and down from the intermediate category of risk (10-20% 10-year risk) after addition of pro-neurotensin to the basic models.

### Example 5

### Association of PNT and Prediction of Breast Cancer in Comparison to Other Cancers in Female and in Comparison to Prediction of Cancer in Males

There is a highly significant association of PNT and prediction of breast cancers. There is no significant association in cervix cancer, uterus cancer and ovary cancer.

This supports the present finding that elevated levels of neurotensin/proneurotensin 1-117 in bodily fluids enhance the risk of getting cancer where neurotensin and/or proneurotensin plays a role, i.e. in cancers where neurotensin receptor is over expressed in said cancer cells.

Further, there is no significant association of PNT and prediction of prostate cancer.

TABLE 10
Association of PNT and prediction of cancer:

| female | | | |
|---|---|---|---|
| No subjects at | | p-value for | |
| baseline: | No of | difference at | |
| 2240 | events | baseline | significance |
| Breast cancer | 128 | 0.001 | +++ |
| Cervix cancer | 18 | 0.997 | −−− |
| Uterus cancer | 18 | 0.366 | −−− |
| Ovary cancer | 15 | 0.94 | −−− |
| male | | | |
| No of subjects | | | |
| at baseline: | | | |
| 1911 | | | |
| Prostate cancer | 163 | 0.310 | −−− |

### FIGURE DESCRIPTION

FIG. 1 shows a typical P-NT dose/signal curve

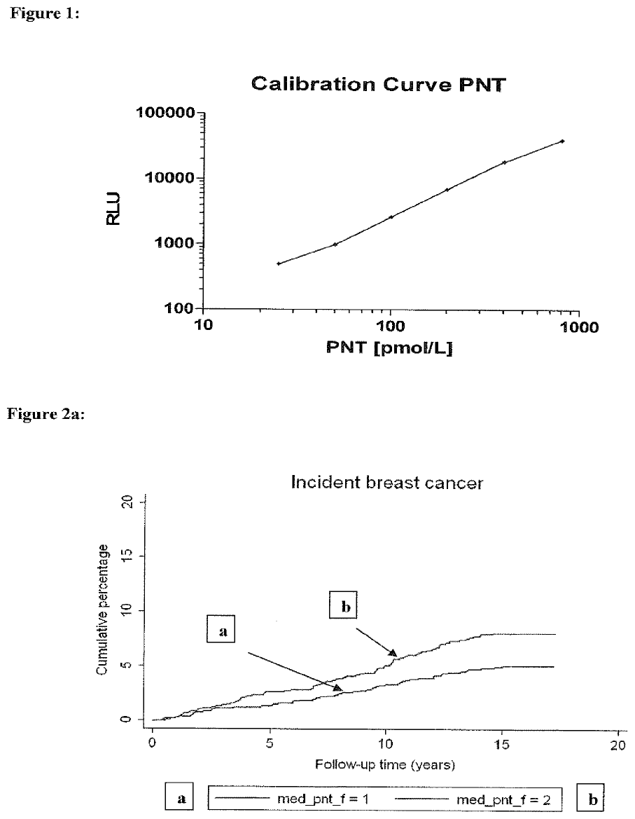

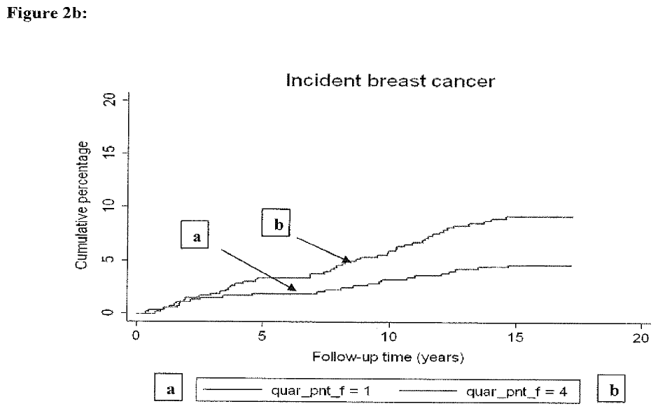

Legend FIGS. 2a and 2b: Increased P-NT indicates a long term increased risk of breast cancer development. Since any women with cancer history at day of baseline (blood sampling) were excluded, PNT is highly predictive for future breast cancer development. Over all, women from Q4 have an 2 times higher risk to develop breast cancer than women from Q 1.

1. A method for predicting the risk of getting cancer in a female subject that does not suffer from cancer in a female subject comprising:
determining the level of pro-neurotensin 1-117 or fragments thereof of at least 5 amino acids or pro-neurotensin 1-117 comprising peptides in a bodily fluid obtained from said female subject; and
correlating said level of pro-neurotensin 1-117 or fragments thereof or pro-neurotensin 1-117 comprising peptides with the risk of getting cancer, wherein an
elevated level is predictive for an enhanced risk of getting cancer and, wherein said cancer is selected from the group comprising breast cancer, lung cancer, pancreatic cancer and colon cancer.

2. A method according to claim 1, wherein said cancer is breast cancer.

3. A method according to claim 1, wherein elevated level” means a level above a threshold level.

4. A method according to claim 1, wherein the level of pro-neurotensin 1-117 or fragments thereof of at least 5 amino acids or pro-neurotensin 1-117 comprising peptides in a bodily fluid is the fasting level.

5. A method according to claim 1, wherein said female subject has never had a history of diagnosis of cancer at the time the sample of bodily fluid is taken from said female subject.

6. A method according to claim 1, wherein said female subject has had a history of diagnosis of cancer and has been cured at the time the sample of bodily fluid is taken from said female subject and the risk of reoccurrence of getting breast cancer is determined.

7. A method according to claim 1, wherein at the time the sample of bodily fluid is taken from said female subject, said female subject has been diagnosed as having a cardiovascular disease or diabetes.

8. A method according to claim 7, wherein at the time the sample of bodily fluid is taken from said female subject, said cardiovascular disease may be selected from the group comprising heart failure, atherosclerosis, and hypertension.

9. A method according to claim 7, wherein at the time the sample of bodily fluid is taken from said female subject, said female subject has been diagnosed as having diabetes type 2.

10. A method according to claim 1, wherein additionally at least one clinical parameter is determined selected from the group comprising: age, presence of diabetes mellitus, current smoking.

11. A method according to claim 1, wherein the level of pro-neurotensin 1-117 is determined.

12. A method according to claim 1, wherein the level of pro-neurotensin 1-117 or fragments thereof or pro-neurotensin 1-117 comprising peptides is measured with an immunoassay.

13. A method according to claim 1 wherein said method is performed more than once in order to monitor the risk of getting breast cancer in a female subject or in order to monitor the course of treatment.

14. A method according to claim 13 wherein said monitoring is performed in order to evaluate the response of said female subject to preventive and/or therapeutic measures taken.

15. A method according to claim 1 in order to stratify said female subjects into risk groups.

16. A method according to claim 15 wherein said female subject is reclassified as a consequence of the determination of the level of pro-neurotensin 1-117 or fragments thereof of at least 5 amino acids or pro-neurotensin 1-117 comprising peptides in a bodily fluid obtained from said female subject.

17. A point-of-care device for performing a method according to claim 1.

18. (canceled)

19. (canceled)

20. (canceled)

21. (canceled)

22. (canceled)


SEQUENCE LISTING (12 sequences)

SEQ ID NO: 1 - protein, 147 aa, homo sapiens
SDSEEEMKALEADFLTNMHTSKISKAHVPSWKMTLLNVCSLVNNLNSPAEETGEVHEEELVARRKLPTALDGFSLEAMLTIYQLHKICHSRAFQHWELIQEDILDTGNDKNGKEEVIKRKIPYILKRQLYENKPRRPYILKRDSYYY

SEQ ID NO: 2 - protein, 125 aa, homo sapiens
SDSEEEMKALEADFLTNMHTSKISKAHVPSWKMTLLNVCSLVNNLNSPAEETGEVHEEELVARRKLPTALDGFSLEAMLTIYQLHKICHSRAFQHWELIQEDILDTGNDKNGKEEVIKRKIPYIL

SEQ ID NO: 3 - protein, 6 aa, homo sapiens
KIPYIL

SEQ ID NO: 4 - protein, 13 aa, homo sapiens
  PEPTIDE (1)..(1): position 1 is pyroQ
QLYENKPRRPYIL

SEQ ID NO: 5 - protein, 117 aa, homo sapiens
SDSEEEMKALEADFLTNMHTSKISKAHVPSWKMTLLNVCSLVNNLNSPAEETGEVHEEELVARRKLPTALDGFSLEAMLTIYQLHKICHSRAFQHWELIQEDILDTGNDKNGKEEVI

SEQ ID NO: 6 - protein, 132 aa, homo sapiens
SDSEEEMKALEADFLTNMHTSKISKAHVPSWKMTLLNVCSLVNNLNSPAEETGEVHEEELVARRKLPTALDGFSLEAMLTIYQLHKICHSRAFQHWELIQEDILDTGNDKNGKEEVIKRKIPYILKRQLYEN

SEQ ID NO: 7 - protein, 125 aa, homo sapiens; identical to SEQ ID NO: 2

SEQ ID NO: 8 - protein, 21 aa, homo sapiens
KIPYILKRQLYENKPRRPYIL

SEQ ID NO: 9 - protein, 28 aa, homo sapiens
KIPYILKRQLYENKPRRPYILKRDSYYY

SEQ ID NO: 10 - protein, 20 aa, homo sapiens
QLYENKPRRPYILKRDSYYY

SEQ ID NO: 11 - protein, 20 aa, Homo sapies
CSDSEEEMKALEADFLTNMH

SEQ ID NO: 12 - protein, 20 aa, homo sapiens
CNLNSPAEETGEVHEEELVA